(19) United States
(12) Patent Application Publication
(10) Pub. No.: US 2024/0091251 A1
(43) Pub. Date: Mar. 21, 2024

(54) COMPOSITIONS COMPRISING AN RNA POLYMERASE INHIBITOR AND CYCLODEXTRIN FOR TREATING VIRAL INFECTIONS

(71) Applicant: Gilead Sciences, Inc., Foster City, CA (US)

(72) Inventors: Nate Larson, Saint George, UT (US); Robert G. Strickley, San Mateo, CA (US)

(21) Appl. No.: 18/241,303

(22) Filed: Sep. 1, 2023

Related U.S. Application Data
(62) Division of application No. 17/585,651, filed on Jan. 27, 2022.
Application No. 17/585,651 is a division of application No. 16/865,209, filed on May 1, 2020, now Pat. No. 11,266,681.
Application No. 16/865,209 is a division of application No. 16/031,620, filed on Jul. 10, 2018, now Pat. No. 10,675,296.
(60) Provisional application No. 62/530,971, filed on Jul. 11, 2017.

(51) Int. Cl. A61K 31/724; A61K 9/00; A61K 9/19; A61K 31/683; A61K 47/40; A61K 47/69; A61P 31/12

(52) U.S. Cl. CPC A61K 31/724; A61K 9/0019; A61K 9/19; A61K 31/683; A61K 47/40; A61K 47/6951; A61P 31/12

(57) ABSTRACT

The present disclosure provides a composition comprising Compound 1, or a pharmaceutically acceptable salt thereof, cyclodextrin, and, optionally, pH adjusting agents.

### CROSS REFERENCE TO RELATED APPLICATIONS

This application is a continuation of U.S. Ser. No. 17/585,651, filed Jan. 27, 2022, which is a divisional of U.S. Ser. No. 16/865,209, filed May 1, 2020, now issued U.S. Pat. No. 11,266,681, which is a divisional of U.S. Ser. No. 16/031,620, filed Jul. 10, 2018, now issued U.S. Pat. No. 10,675,296, which claim priority to U.S. Provisional Application No. 62/530,971, filed Jul. 11, 2017, and each application is hereby incorporated by reference in its entirety for all purposes.

### FIELD

Provided are pharmaceutical compositions suitable for treating viral infections such as Arenaviridae, Coronaviridae, Filoviridae, Flaviviridae, or Paramyxoviridae viral infections. In particular, formulations comprising Compound 1, or a pharmaceutically acceptable salt thereof, cyclodextrin and, optionally, pH adjusting agents.

### BACKGROUND

Preventing or treating some Arenaviridae, Coronaviridae, Filoviridae, Flaviviridae, and Paramyxoviridae viral infections present challenges due to a lack of vaccine or post-exposure treatment modality for preventing or managing infections caused by viruses from these families. In some cases, patients only receive supportive therapy such as electrolyte and fluid balancing, oxygen, blood pressure maintenance, or treatment for secondary infections.

The compound (S)-2-ethylbutyl 2-(((S)-(((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy) phosphoryl)amino)propanoate, referred to herein as Compound 1, is known to exhibit antiviral properties against Arenaviridae, Coronaviridae, Filoviridae, and Paramyxoviridae viruses as described in Warren, T. et al., Nature (2016) 531:381-385, and antiviral activities against Flaviviridae viruses as described in co-pending International Publication No. WO 2017/184668. There is a need to administer Compound 1 parenterally to certain patients, however, Compound 1 is relatively insoluble and chemically unstable in aqueous media, thus there is a need to develop a composition comprising Compound 1, or a pharmaceutically acceptable salt thereof, that exhibits improved solubility, improved usability for parenteral administration, and sufficient room-temperature and elevated temperature stability to avoid the use of cold-chain for transport and/or storage.

Certain solubilizers may be used to improve the solubility of a compound to form a composition capable of being administered parenterally, however such solubilizers may have certain undesirable effects (Stella, et. al. Toxicologic Pathology (2008), Vol 36, Number 1, pages 30-42). For example, a formulation including polysorbate 80 may have potential hemodynamic effects, tubing limitations, extractables and leachables from tubing, limitations on stoppers, potential for precipitation upon dilution, or issues with adaptability for pediatric use. As another example, beta-cyclodextrin derivatives are known to have certain physiological effects on kidneys, thus there is also a need to limit the amount of such solubilizers in a pharmaceutical formulation. The present disclosure provides a composition comprising Compound 1, or a pharmaceutically acceptable salt thereof, that exhibits improved solubility and/or improved usability for parenteral administration and limits the amount of beta-cyclodextrin derivatives.

### SUMMARY

The present disclosure provides a composition comprising (S)-2-ethylbutyl 2-(((S)-(((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy) phosphoryl)amino)propanoate (Compound 1), or a pharmaceutically acceptable salt thereof, cyclodextrin, and, optionally, pH adjusting agents.

The present disclosure provides a composition comprising

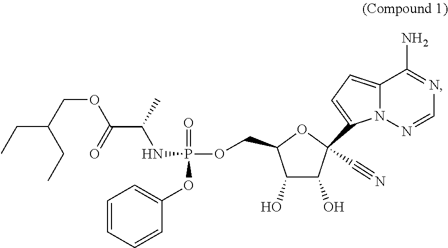 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

or a pharmaceutically acceptable salt thereof, cyclodextrin, and, optionally, pH adjusting agents.

### DESCRIPTION OF THE FIGURES

FIG. 1: pH Stability for Compound 1.

FIG. 2: pH Solubility Profile for Compound 1.

### DETAILED DESCRIPTION

### I. General

The present invention includes a composition of Compound 1 and a cyclodextrin, such as betadex-sulfobutylether sodium, that is surprisingly stable at room temperature. The composition is substantially free of water following a dehydration or lyophilization process. The lyophilized composition can be prepared by suspending a crystalline form of Compound 1 in an acidic solution of betadex-sulfobutylether sodium, and subsequently adjusting the pH to between 3 to 4, to produce a solution that surprisingly maintains the complexation between Compound 1 and the betadex-sulfobutylether sodium, and lyophilizing the solution. The neutralized mixture is then lyophilized.

### II. Definitions

(S)-2-Ethylbutyl 2-(((S)-(((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy) phosphoryl)amino) propanoate (Compound 1). Compound 1 is a viral RNA polymerase inhibitor with antiviral properties against Arenaviridae, Coronaviridae, Filoviridae, Flaviviridae, and Paramyxoviridae viruses. It has the following formula, as disclosed in PCT Publication No. WO2016/069826:

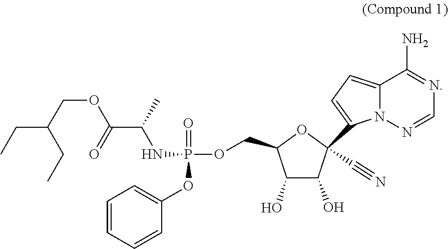 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

The IUPAC name for Compound 1 is (S)-2-ethylbutyl 2-(((S)-(((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy) phosphoryl)amino)propanoate. The CAS Registry Number for Compound 1 is 1809249-37-3.

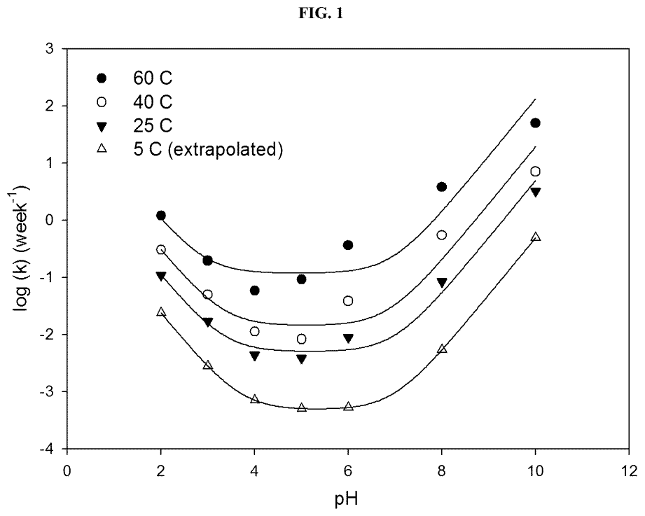

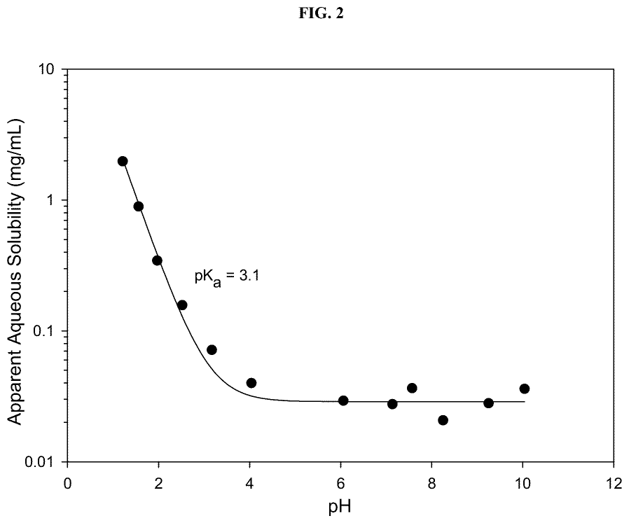

The stability of Compound 1 was determined by monitoring the concentration of Compound 1 in solution by liquid chromatography at various storage conditions. The stability results are shown in FIG. 1. Compound 1 is most stable in solution between about pH 4 and 5. The solubility of Compound 1 between pH 1 and 10 was determined by quantitatively measuring the concentration of Compound 1 in solution at various pH conditions by liquid chromatography. The solubility results are shown in FIG. 2. The solubility of Compound 1 increases at or below pH 4.

Cyclodextrin. Cyclodextrin is a chemical family of cyclic compound typically having 6, 7, or 8 sugar units. A cyclodextrin comprising 6 sugar units is an alpha-cyclodextrin (α-cyclodextrin). A cyclodextrin comprising 7 sugar units is a beta-cyclodextrin (β-cyclodextrin). A cyclodextrin comprising 8 sugar units is a gamma-cyclodextrin (γ-cyclodextrin).

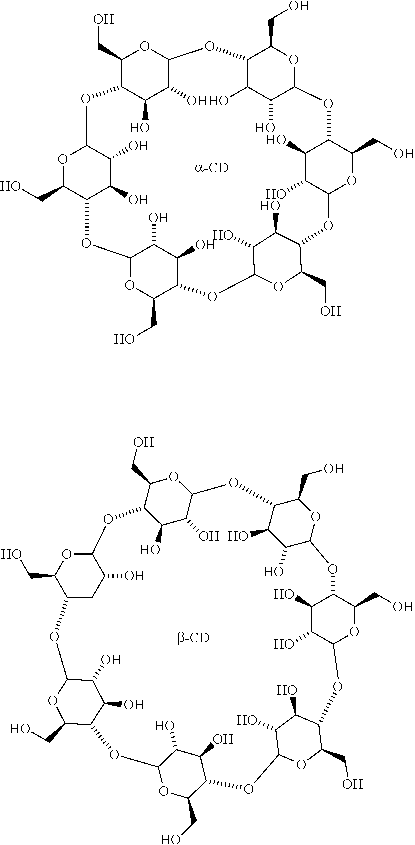

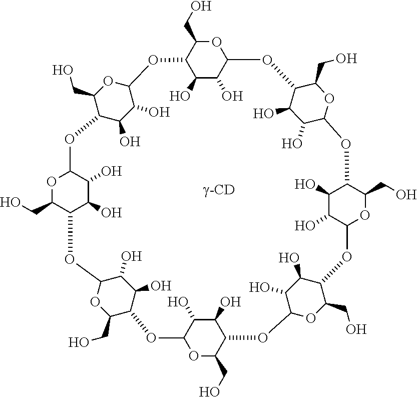

Cyclodextrin derivatives are cyclodextrins where some of the —OH groups are modified to —OR groups. Non-limiting examples of cyclodextrin derivatives include cyclodextrins where —OH groups are modified to —OR wherein each R is independently alkyl, hydroxyalkyl, glucosyl or maltosyl groups, or —(CH₂)₄SO₃⁻Na⁺.

Non-limiting examples of commercial cyclodextrin derivatives include CAPTISOL®, CAVITRON®, DEXOLVE-7®, and KLEPTOSE®. CAPTISOL® (herein referred to as Captisol) is a registered trademark of Ligand Corporation. Captisol refers to sulfobutylalkylether-beta-cyclodextrin sold by or licensed by Ligand Pharmaceuticals. CAVITRON® (herein referred to as Cavitron) is a registered trademark of Wacker Chemie AG. Cavitron is an excipient obtained by the substitution of hydroxyl groups on native cyclodextrins to make hydroxypropyl-beta-cyclodextrins (HPBCD), a process that significantly enhance their solubility and makes them more suitable for drug solubilization. DEXOLVE-7® (herein referred to as Dexolve-7) is a registered trademark of CycloLabs Limited. Dexolve-7 is sulfobutylalkylether-beta-cy clodextrin sodium salt, an excipient used in pharmaceutical formulations to improve solubility. KLEPTOSE® (herein referred to as Kleptose) is a registered trademark of Roquette Pharmaceuticals, Geneva, Illinois, USA. Kleptose is a brand of hydroxypropyl-beta-cyclodextrin.

In some embodiments, “cyclodextrin” refers to beta-cyclodextrin derivatives selected from the group consisting of sulfobutylalkylether-beta-cyclodextrin, betadex-sulfobutylether sodium, and hydroxypropyl-beta-cyclodextrin. In some embodiments, “cyclodextrin” refers to sulfobutylalkylether-beta-cyclodextrin. In some embodiments, “cyclodextrin” refers to betadex-sulfobutylether sodium. In some embodiments, “cyclodextrin” refers to hydroxypropyl-beta-cyclodextrin. In some embodiments, “cyclodextrin” refers to the formula

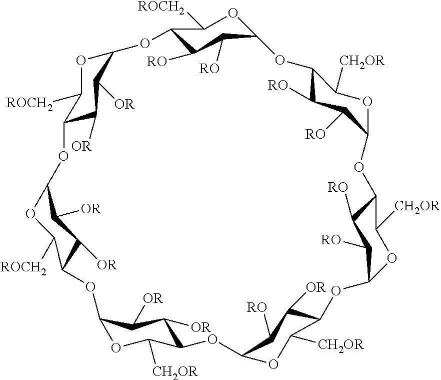

wherein R is —H or CH₂CH₂CH₂CH₂SO₃⁻Na⁺.

pH Adjusting Agent. pH adjusting agents are buffers, acids, and bases. “Acid” refers to a compound that is capable of donating a proton (H⁺) under the Bronsted-Lowry definition, or is an electron pair acceptor under the Lewis definition. Acids useful in the present invention are Bronsted-Lowry acids that include, but are not limited to, alkanoic acids or carboxylic acids (formic acid, acetic acid, citric acid, lactic acid, oxalic acid, etc.), sulfonic acids and mineral acids, as defined herein. Mineral acids are inorganic acids such as hydrogen halides (hydrofluoric acid, hydrochloric acid, hydrobromice acid, etc.), halogen oxoacids (hypochlorous acid, perchloric acid, etc.), as well as sulfuric acid, nitric acid, phosphoric acid, chromic acid and boric acid. Sulfonic acids include methanesulfonic acid, benzenesulfonic acid, p-toluenesulfonic acid, trifouromethanesulfonic acid, camphorsulfonic acid, among others. “Base” refers to a compound capable of accepting a proton (H⁺) under the Bronsted-Lowry definition, or is an electron-pair donor under the Lewis definition. Bases useful in the present invention that are Bronsted-Lowry bases include hydroxides such as lithium hydroxide, sodium hydroxide, potassium hydroxide, rubidium hydroxide, cesium hydroxide, magnesium hydroxide, strontium hydroxide, barium hydroxide, and others. Lewis bases include, but are not limited to, amines such as ammonia, trimethylamine, triethylamine, diisopropylethylamine (DIPEA or Hunig's Base), 1,8-diazabicycloundec-7-ene (DBU), 2,6-di-tert-butylpyridine, quinuclidine, and lithium di-isopropylamine (LDA), and nucleophilic bases such as butyl-lithium. Other bases are known to one of skill in the art. In some embodiments, the pH adjusting agents are NaOH and HCl. In some embodiments, the pH adjusting agent is NaOH. In some embodiments, the pH adjusting agent is HCl.

“Dehydrate” refers to the process of removing water from a sample via evaporation, sublimation, or a combination thereof. Evaporation refers to the transition of a substance from a liquid state to a gaseous state, and sublimation to the transition from a solid state directly to a gaseous state. Dehydration can occur at a variety of temperatures and pressures. When dehydration occurs below the freezing point of the sample, this is referred to as freeze-drying or lyophilizing, the process of removing water from a sample at low temperature and pressure.

The term “treatment” or “treating” means any administration of Compound 1 according to the present disclosure to a subject (e.g. human) having or susceptible to a condition or disease disclosed herein for the purpose of 1) preventing or protecting against the disease or condition, that is, causing the clinical symptoms not to develop; 2) inhibiting the disease or condition, that is, arresting or suppressing the development of clinical symptoms; or 3) relieving the disease or condition that is causing the regression of clinical symptoms. In some embodiments, the term “treatment” or “treating” refers to relieving the disease or condition, i.e. which is causing the regression of clinical symptoms.

As used herein, the term “preventing” refers to the prophylactic treatment of a patient in need thereof. The prophylactic treatment can be accomplished by providing an appropriate dose of a therapeutic agent e.g. Compound 1, to a subject at risk of suffering from an ailment, thereby substantially averting onset of the ailment. The presence of a genetic mutation or the predisposition to having a mutation may not be alterable. However, prophylactic treatment (prevention) as used herein has the potential to avoid/ameliorate the symptoms or clinical consequences of having the disease engendered by such genetic mutation or predisposition.

“Subject” refers to animals such as mammals, including, but not limited to, primates (e.g., humans), cows, sheep, goats, horses, dogs, cats, rabbits, rats, mice and the like. In certain embodiments, the subject is a human.

The term “therapeutically effective amount”, as used herein, is the amount of Compound 1, or a pharmaceutically acceptable salt thereof, present in a composition described herein that is needed to provide a desired level of drug in the secretions and tissues of the airways and lungs, or alternatively, in the bloodstream of a subject to be treated to give an anticipated physiological response or desired biological effect when such a composition is administered by the chosen route of administration. The precise amount will depend upon numerous factors, for example the specific activity of the composition, the delivery device employed, the physical characteristics of the composition, its intended use, as well as animal considerations such as severity of the disease state, veterinarian cooperation, etc., and can readily be determined by one skilled in the art based upon the information provided herein.

“Safe water for injection”, “water safe for infection” or “sterile water for injection” refers to a water solution for combining with one or more drugs requiring dilution or dissolution prior to administration to the subject. The water for injection is sterile and stored in a glass vial of Type I or II borosilicate glass, or other suitable container. The water for injection does not include any other components, such as stabilizers, antimicrobial agents, buffer, etc.

The term “normal saline” means a water solution containing 0.9% (w/v) NaCl.

The term “hypertonic saline” means a water solution containing greater than 0.9% (w/v) NaCl. For example, 3% hypertonic saline would contain 3% (w/v) NaCl.

“Forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

### III. Formulations

All compositions described here contain Compound 1, or a pharmaceutically acceptable salt thereof, cyclodextrin, and, optionally, pH adjusting agents.

In some embodiments, a composition comprising Compound 1, or a pharmaceutically acceptable salt thereof, cyclodextrin, and, optionally, pH adjusting agents.

In some embodiments, a composition comprising

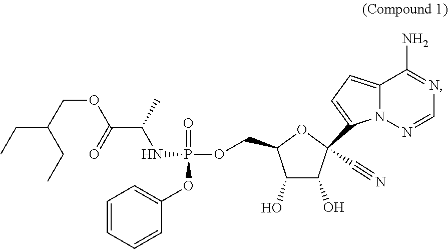 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

or a pharmaceutically acceptable salt thereof, cyclodextrin, and, optionally, pH adjusting agents.

In some embodiments, a composition comprising Compound 1, or a pharmaceutically acceptable salt thereof, and beta-cyclodextrin, and, optionally, pH adjusting agents.

In some embodiments, an antiviral composition comprising Compound 1, or a pharmaceutically acceptable salt thereof, and beta-cyclodextrin, and, optionally, pH adjusting agents.

In some embodiments, a composition comprising Compound 1, or a pharmaceutically acceptable salt thereof, and beta-cyclodextrin, and, optionally, pH adjusting agents, wherein the beta cyclodextrin is sulfobutylalkylether-beta-cyclodextrin, betadex-sulfobutylether sodium, or hydroxypropyl-beta-cyclodextrin.

In some embodiments, a composition comprising Compound 1, or a pharmaceutically acceptable salt thereof, and beta-cyclodextrin, and, optionally, pH adjusting agents, wherein the beta cyclodextrin is betadex-sulfobutylether sodium.

### Solution Composition

In some embodiments, the composition also includes water to form a solution composition. The water can be any suitable water, such as distilled water or water safe for injection. In some embodiments, the water can be water safe for injection.

In some embodiments, a composition comprising Compound 1, or a pharmaceutically acceptable salt thereof, and beta-cyclodextrin, and, optionally, pH adjusting agents, wherein the pH adjusting agents are NaOH and HCl.

Any suitable number of pH adjusting agents can be used in the composition of the present invention. For example, the composition can includes, 1, 2, 3, 4 or more pH adjusting agents. In some embodiments, the composition includes at least one pH adjusting agent. In some embodiments, the composition includes at least 2 pH adjusting agents. When more than 1 pH adjusting agent is used, the pH adjusting agents can be acids, bases, or a mixture thereof. In some embodiments, a first pH adjusting agent includes an acid, and a second pH adjusting agent includes a base.

In some embodiments, the solution composition includes Compound 1, or a pharmaceutically acceptable salt thereof, beta-cyclodextrin, and at least one pH adjusting agent. In some embodiments, the solution composition includes Compound 1, or a pharmaceutically acceptable salt thereof, beta-cyclodextrin, and at least two pH adjusting agent. In some embodiments, the solution composition includes Compound 1, or a pharmaceutically acceptable salt thereof, beta-cyclodextrin, and the pH adjusting agents HCl and NaOH.

The solution composition has a pH of less than 7, preferably less than about 6, 5, or 4. The pH of the solution composition can be from 1 to 6, 2 to 5, or 3 to 4. For example, the pH of the solution composition can be about 3.1, 3.2, 3.3, 3.4, 3.5, 3.6, 3.7, 3.8, or about 3.9. In some embodiments, the solution composition has a pH of from 3 to 4. In some embodiments, the solution composition has a pH of about 3.5.

In some embodiments, an antiviral composition comprising Compound 1, or a pharmaceutically acceptable salt thereof, and beta-cyclodextrin, and, optionally, pH adjusting agents, wherein the pH adjusting agents are NaOH and HCl.

In some embodiments, a composition comprising 90 mg to 175 mg of Compound 1, or a pharmaceutically acceptable salt thereof, and beta-cyclodextrin, and, optionally, pH adjusting agents. In some embodiments, a composition comprising 90 mg to 110 mg of Compound 1, or a pharmaceutically acceptable salt thereof, and beta-cyclodextrin, and, optionally, pH adjusting agents. In some embodiments, a composition comprising 145 mg to 165 mg of Compound 1, or a pharmaceutically acceptable salt thereof, and beta-cyclodextrin, and, optionally, pH adjusting agents. In some embodiments, a composition comprising 100 mg of Compound 1, or a pharmaceutically acceptable salt thereof, and beta-cyclodextrin, and, optionally, pH adjusting agents. In some embodiments, a composition comprising 150 mg of Compound 1, or a pharmaceutically acceptable salt thereof, and beta-cyclodextrin, and, optionally, pH adjusting agents.

In some embodiments, a composition comprising 90 mg to 175 mg of Compound 1, or a pharmaceutically acceptable salt thereof, and beta-cyclodextrin, and, optionally, pH adjusting agents, wherein the pH adjusting agents are NaOH and HCl. In some embodiments, a composition comprising 90 mg to 110 mg of Compound 1, or a pharmaceutically acceptable salt thereof, and beta-cyclodextrin, and, optionally, pH adjusting agents, wherein the pH adjusting agents are NaOH and HCl. In some embodiments, a composition comprising 145 mg to 165 mg of Compound 1, or a pharmaceutically acceptable salt thereof, and beta-cyclodextrin, and, optionally, pH adjusting agents, wherein the pH adjusting agents are NaOH and HCl. In some embodiments, a composition comprising 100 mg of Compound 1, or a pharmaceutically acceptable salt thereof, and beta-cyclodextrin, and, optionally, pH adjusting agents, wherein the pH adjusting agents are NaOH and HCl. In some embodiments, a composition comprising 150 mg of Compound 1, or a pharmaceutically acceptable salt thereof, and beta-cyclodextrin, and, optionally, pH adjusting agents, wherein the pH adjusting agents are NaOH and HCl.

In some embodiments, the cyclodextrin is present at about 5% to 30% w/v. In some embodiments, the cyclodextrin is present at about 10% to 25% w/v. In some embodiments, the cyclodextrin is present at about 14% to 21% w/v. In some embodiments, the cyclodextrin is present at about 15% w/v. In some embodiments, the cyclodextrin is present at about 20% w/v. In some embodiments, the cyclodextrin is present at 15% w/v. In some embodiments, the cyclodextrin is present at 20% w/v.

In some embodiments, the beta-cyclodextrin is present at about 5% to 30% w/v. In some embodiments, the beta-cyclodextrin is present at about 10% to 25% w/v. In some embodiments, the beta-cyclodextrin is present at about 14% to 21% w/v. In some embodiments, the beta-cyclodextrin is present at about 15% w/v. In some embodiments, the beta-cyclodextrin is present at about 20% w/v. In some embodiments, the beta-cyclodextrin is present at 15% w/v. In some embodiments, the beta-cyclodextrin is present at 20% w/v.

In some embodiments, Compound 1 is present at about 1.0 to 10.0 mg/mL. In some embodiments, Compound 1 is present at about 4.0 to 8.0 mg/mL. In some embodiments, Compound 1 is present at about 5.0 to 7.0 mg/mL. In some embodiments, Compound 1 is present at about 5.0 mg/mL. In some embodiments, Compound 1 is present at about 6.7 mg/mL. In some embodiments, Compound 1 is present at 5.0 mg/mL. In some embodiments, Compound 1 is present at 6.7 mg/mL.

In some embodiments, a composition comprising Compound 1, or a pharmaceutically acceptable salt thereof, and beta-cyclodextrin, and, optionally, pH adjusting agents, wherein Compound 1 is present at about 4.0 to 8.0 mg/mL and the beta-cyclodextrin is present at about 5% to 30% w/v. In some embodiments, a composition comprising Compound 1, or a pharmaceutically acceptable salt thereof, and beta-cyclodextrin, and, optionally, pH adjusting agents, wherein Compound 1 is present at about 4.0 to 8.0 mg/mL and the beta-cyclodextrin is present at about 10% to 25% w/v. In some embodiments, a composition comprising Compound 1, or a pharmaceutically acceptable salt thereof, and beta-cyclodextrin, and, optionally, pH adjusting agents, wherein Compound 1 is present at about 4.0 to 8.0 mg/mL and the beta-cyclodextrin is present at about 14% to 21% w/v.

In some embodiments, a composition comprising Compound 1, or a pharmaceutically acceptable salt thereof, and beta-cyclodextrin, and, optionally, pH adjusting agents, wherein Compound 1 is present at about 5.0 to 7.0 mg/mL and the beta-cyclodextrin is present at about 5% to 30% w/v. In some embodiments, a composition comprising Compound 1, or a pharmaceutically acceptable salt thereof, and beta-cyclodextrin, and, optionally, pH adjusting agents, wherein Compound 1 is present at about 5.0 to 7.0 mg/mL and the beta-cyclodextrin is present at about 10% to 25% w/v. In some embodiments, a composition comprising Compound 1, or a pharmaceutically acceptable salt thereof, and beta-cyclodextrin, and, optionally, pH adjusting agents, wherein Compound 1 is present at about 5.0 to 7.0 mg/mL and the beta-cyclodextrin is present at about 14% to 21% w/v.

In some embodiments, a composition comprising Compound 1, or a pharmaceutically acceptable salt thereof, and beta-cyclodextrin, and, optionally, pH adjusting agents, wherein Compound 1 is present at about 5.0 mg/mL and the beta-cyclodextrin is present at about 15% w/v. In some embodiments, a composition comprising Compound 1, or a pharmaceutically acceptable salt thereof, and beta-cyclodextrin, and, optionally, pH adjusting agents, wherein Compound 1 is present at about 6.7 mg/mL and the beta-cyclodextrin is present at about 20% w/v.

In some embodiments, a composition comprising Compound 1, or a pharmaceutically acceptable salt thereof, and beta-cyclodextrin, and, optionally, pH adjusting agents, wherein Compound 1 is present at about 5.0 mg/mL and the beta-cyclodextrin is present at about 15% w/v further comprising water for injection. In some embodiments, a composition comprising Compound 1, or a pharmaceutically acceptable salt thereof, and beta-cyclodextrin, and, optionally, pH adjusting agents, wherein Compound 1 is present at about 6.7 mg/mL and the beta-cyclodextrin is present at about 20% w/v further comprising water for injection.

In some embodiments, a vial comprising Compound 1, or a pharmaceutically acceptable salt thereof, and beta-cyclodextrin, and, optionally, pH adjusting agents, wherein Compound 1 is present at about 5.0 mg/mL and the beta-cyclodextrin is present at about 15% w/v further comprising water for injection. In some embodiments, a vial comprising Compound 1, or a pharmaceutically acceptable salt thereof, and beta-cyclodextrin, and, optionally, pH adjusting agents, wherein Compound 1 is present at about 6.7 mg/mL and the beta-cyclodextrin is present at about 20% w/v further comprising water for injection.

In some embodiments, a lyophilized composition comprising Compound 1, or a pharmaceutically acceptable salt thereof, and beta-cyclodextrin, and, optionally, pH adjusting agents, wherein Compound 1 is present at about 5.0 mg/mL and the beta-cyclodextrin is present at about 15% w/v. In some embodiments, a lyophilized composition comprising Compound 1, or a pharmaceutically acceptable salt thereof, and beta-cyclodextrin, and, optionally, pH adjusting agents, wherein Compound 1 is present at about 6.7 mg/mL and the beta-cyclodextrin is present at about 20% w/v. In some embodiments, a lyophilized composition comprising about 90 to 175 mg Compound 1, or a pharmaceutically acceptable salt thereof, and beta-cyclodextrin, and, optionally, pH adjusting agents, wherein the beta-cyclodextrin is present at about 15 to 20% w/v. In some embodiments, a lyophilized composition comprising about 100 mg Compound 1, or a pharmaceutically acceptable salt thereof, and beta-cyclodextrin, and, optionally, pH adjusting agents, wherein the beta-cyclodextrin is present at about 15 to 20% w/v. In some embodiments, a lyophilized composition comprising about 150 mg Compound 1, or a pharmaceutically acceptable salt thereof, and beta-cyclodextrin, and, optionally, pH adjusting agents, wherein the beta-cyclodextrin is present at about 15 to 20% w/v.

In some embodiments, a composition comprising crystalline Compound 1, or a pharmaceutically acceptable salt thereof, and beta-cyclodextrin, and, optionally, pH adjusting agents, wherein crystalline Compound 1 is characterized by an X-ray powder diffraction (XRPD) pattern having at least three peaks selected from the group consisting of 22.3°, 16.2°, 22.5°, 13.8°, 12.7°, 16.9°, 10.6°, 14.5°, 24.3, 24.0°, 17.6°, 23.4°, 8.1°, 11.0°, 26.8°, 28.9°, 19.6°, 27.8°, 26.4°, 28.7°, 29.8°, 33.0°, 18.8°, 18.3°, 32.1°, 25.3°, 32.6°, 8.6°, 34.2°, 35.9°, 27.2°, 28.1°, 38.9°, 34.6°, 17.1°, 35.2°, 21.4°, 30.6°, 25.6°, 18.5°, 31.7°, 36.5°, and 37.1°+0.2° 2-0.

### Lyophilized Composition

The composition of the present invention also includes a lyophilized or dehydrated composition of Compound 1, or a pharmaceutically acceptable salt thereof, and cyclodextrin. In some embodiments, the present invention provides a lyophilized composition including Compound 1, or a pharmaceutically acceptable salt thereof, and cyclodextrin. The lyophilized composition can be in any suitable solid form, such as a powder.

Compound 1 can be present in the lyophilized composition in an amount from 1% to 10% (w/w), or from 1 to 5%, or from 2 to 4%, or from 3 to 4%, or from 3 to 3.5% (w/w). In some embodiments, the lyophilized composition includes Compound 1 in an amount from 1% to 10% (w/w). In some embodiments, the lyophilized composition include Compound 1 in an amount from 1% to 5% (w/w). In some embodiments, the lyophilized composition includes Compound 1 in an amount from 2% to 4% (w/w). In some embodiments, the lyophilized composition includes Compound 1 in an amount from 3% to 3.5% (w/w).

Compound 1 can be present in the lyophilized composition in an amount of about 1% (w/w), or 1.5, 2, 2.1, 2.2, 2.3, 2.4, 2.5, 2.6, 2.7, 2.8, 3.9, 3, 3.1, 3.2, 3.3, 3.4, 3.5, 3.6, 3.7, 3.8, 3.9, 4, 4.5, 5, 6, 7, 8, 9, or about 10% (w/w). In some embodiments, the lyophilized composition includes Compound 1 at about 3.2% (w/w).

Cyclodextrin can be present in the lyophilized composition in an amount from 90% to 99% (w/w), or from 95 to 99%, or from 96 to 98%, or from 96.5 to 97% (w/w). In some embodiments, the lyophilized composition includes cyclodextrin in an amount from 90% to 99% (w/w). In some embodiments, the lyophilized composition includes cyclodextrin in an amount from 95 to 99% (w/w). In some embodiments, the lyophilized composition includes cyclodextrin in an amount from 96 to 98% (w/w). In some embodiments, the lyophilized composition includes cyclodextrin in an amount from 96.5 to 97% (w/w).

Cyclodextrin can be present in the lyophilized composition in an amount of about 90% (w/w), or 91, 92, 93, 94, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, or about 99% (w/w). In some embodiments, the lyophilized composition includes cyclodextrin at about 96.8% (w/w). In some embodiments, the lyophilized composition includes betadex-sulfobutylether sodium at about 96.8% (w/w). In some embodiments, the lyophilized composition includes Compound 1 at about 3.2% (w/w), and cyclodextrin at about 96.8% (w/w). In some embodiments, the lyophilized composition includes Compound 1 at about 3.2% (w/w), and betadex-sulfobutylether sodium at about 96.8% (w/w).

The lyophilized composition of the present invention can include Compound 1 in an amount from b % to 10% (w/w), or from 1 to 5%, or from 2 to 4%, or from 3 to 4%, or from 3 to 3.5%, and cyclodextrin in an amount from 90% to 99% (w/w), or from 95 to 99%, or from 96 to 98%, or from 96.5 to 97% (w/w). In some embodiments, the lyophilized composition includes Compound 1 in an amount from 1% to 10% (w/w), and cyclodextrin in an amount from 90% to 99% (w/w). In some embodiments, the lyophilized composition include Compound 1 in an amount from 1% to 5% (w/w), and cyclodextrin in an amount from 95 to 99% (w/w). In some embodiments, the lyophilized composition includes Compound 1 in an amount from 2% to 4% (w/w), and cyclodextrin in an amount from 96 to 98% (w/w). In some embodiments, the lyophilized composition includes Compound 1 in an amount from 3% to 3.5% (w/w), and cyclodextrin in an amount from 96.5 to 97% (w/w).

In some embodiments, the lyophilized composition includes Compound 1 at about 3.2% (w/w) and cyclodextrin in an amount of about 96.8% (w/w). In some embodiments, the lyophilized composition includes Compound 1 at 3.2% (w/w) and cyclodextrin in an amount of 96.8% (w/w). In some embodiments, the lyophilized composition consists essentially of Compound 1 at about 3.2% (w/w) and cyclodextrin in an amount of about 96.8% (w/w). In some embodiments, the lyophilized composition consists essentially of Compound 1 at 3.2% (w/w) and cyclodextrin in an amount of 96.8% (w/w).

In some embodiments, the lyophilized composition includes Compound 1 at about 3.2% (w/w) and betadex-sulfobutylether sodium in an amount of about 96.8% (w/w). In some embodiments, the lyophilized composition includes Compound 1 at 3.2% (w/w) and betadex-sulfobutylether sodium in an amount of 96.8% (w/w). In some embodiments, the lyophilized composition consists essentially of Compound 1 at about 3.2% (w/w) and betadex-sulfobutylether sodium in an amount of about 96.8% (w/w). In some embodiments, the lyophilized composition consists essentially of Compound 1 at 3.2% (w/w) and betadex-sulfobutylether sodium in an amount of 96.8% (w/w).

The cyclodextrin of the lyophilized composition can include any suitable cyclodextrin as described above. For example, the cyclodextrin can be a beta-cyclodextrin, such as sulfobutylalkylether-beta-cyclodextrin, betadex-sulfobutylether sodium, or hydroxypropyl-beta-cyclodextrin. In some embodiments, the lyophilized composition includes a beta-cyclodextrin. In some embodiments, the lyophilized composition includes sulfobutylalkylether-beta-cyclodextrin, betadex-sulfobutylether sodium, or hydroxypropyl-beta-cyclodextrin. In some embodiments, the lyophilized composition includes betadex-sulfobutylether sodium.

In some embodiments, a composition comprising Compound 1, or a pharmaceutically acceptable salt thereof, and beta-cyclodextrin, and, optionally, pH adjusting agents, wherein Compound 1 is 3%±1% w/w and beta-cyclodextrin is 97%±1% w/w. In some embodiments, a composition comprising Compound 1, or a pharmaceutically acceptable salt thereof, and beta-cyclodextrin, and, optionally, pH adjusting agents, wherein Compound 1 is 3%±0.5% w/w and beta-cyclodextrin is 97%±0.5% w/w. In some embodiments, a composition comprising Compound 1, or a pharmaceutically acceptable salt thereof, and beta-cyclodextrin, and, optionally, pH adjusting agents, wherein Compound 1 is about 3.2% w/w and beta-cyclodextrin is 96.8% w/w.

The lyophilized compositions of the present invention are surprisingly stable, showing relatively little impurity formation when tested at raised temperatures and relative humidity. For example, the purity of the lyophilized composition can be at least 95%, or 96, 97, 98, 99, 99.1, 99.2, 99.3, 99.4, 99.5, 99.6, 99.7, 99.8, or at least 99.9%. Thus, the lyophilized composition can have an impurity in the amount of less than 5%, or 4, 3, 2, 1, 0.9, 0.8, 0.7, 0.6, 0.5, 0.4, 0.3, 0.2, or less than 0.1%. In some embodiments, the lyophilized composition has less than about 1% AN via HPLC of impurity following one or more of the following: (i) storage for 18 months at a temperature of about 25° C. (±1° C.) and a relative humidity of about 60% (±1%); (ii) storage for 18 months at a temperature of about 30° C. (±1° C.) and a relative humidity of about 75% (±1%); or (iii) storage for 6 months at a temperature of about 40° C. (±1° C.) and a relative humidity of about 75% (±1%). In some embodiments, the lyophilized composition has less than about 0.5% AN via HPLC of impurity following one or more of the following: (i) storage for 18 months at a temperature of about 25° C. (±1° C.) and a relative humidity of about 60% (±1%); (ii) storage for 18 months at a temperature of about 30° C. (±1° C.) and a relative humidity of about 75% (±1%); or (iii) storage for 6 months at a temperature of about 40° C. (±1° C.) and a relative humidity of about 75% (±1%).

The lyophilized composition can include various forms of Compound 1. For example, Compound 1 can be amorphous or crystalline, or a mixture thereof. In some embodiments, the lyophilized composition includes amorphous Compound 1.

The lyophilized composition can be contained in any suitable container, such as a sealed vial. In some embodiments, the present invention provides a sealed vial containing the lyophilized composition. In some embodiments, the present invention provides a sealed vial containing the lyophilized composition consisting essentially of Compound 1 at 3.2% (w/w) and betadex-sulfobutylether sodium in an amount of 96.8% (w/w).

### Injectable Composition

The composition of the present invention also includes an injectable composition for administration to subjects and patients in need of treatment. The injectable composition includes the lyophilized composition described above, and water. In some embodiments, the present invention provides an injectable composition, including Compound 1:

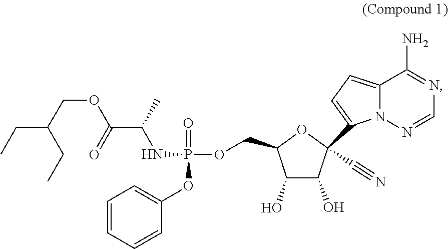 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

or a pharmaceutically acceptable salt thereof, in an amount from 0.1% to 10% w/v; cyclodextrin in an amount from 10% to 50% w/v; and water.

The cyclodextrin of the injectable composition can include any suitable cyclodextrin as described above. For example, the cyclodextrin can be a beta-cyclodextrin, such as sulfobutylalkylether-beta-cyclodextrin, betadex-sulfobutylether sodium, or hydroxypropyl-beta-cyclodextrin. In some embodiments, the injectable composition includes a beta-cyclodextrin. In some embodiments, the injectable composition includes sulfobutylalkylether-beta-cyclodextrin, betadex-sulfobutylether sodium, or hydroxypropyl-beta-cyclodextrin. In some embodiments, the injectable composition includes betadex-sulfobutylether sodium.

The water of the injectable composition can be any suitable type of water. For example, the water of the injectable composition can be water safe for injection.

The injectable composition include any suitable amount of Compound 1 from 0.1% to 10% w/v. For example, Compound 1 can be present in the injectable composition in an amount from 0.10% to 5% w/v, 0.1 to 4, 0.1 to 3, 0.1 to 2, 0.1 to 1, 0.2 to 0.8, 0.3 to 0.7, or 0.4% to 0.6% w/v. Compound 1 can be present in the injectable composition in an amount of about 0.1% w/v, or 0.2, 0.3, 0.4, 0.5, 0.6, 0.7, 0.8, 0.9, or about 1% w/v. In some embodiments, Compound 1 is present in the injectable composition in an amount of 0.1% to 10% w/v. In some embodiments, Compound 1 is present in the injectable composition in an amount of 0.1% to 1% w/v. In some embodiments, Compound 1 is present in the injectable composition in an amount of about 0.5% w/v.

The injectable composition include any suitable amount of Compound 1 from 0.1 to 100 mg/mL. For example, Compound 1 can be present in the injectable composition in an amount from 0.1 to 100 mg/mL, or 0.1 to 50, 0.5 to 10, 1 to 10, 2 to 8, 3 to 7, 4 to 6, or 4.5 to 5.5 mg/mL. Compound 1 can be present in the injectable composition in an amount of about 0.1 mg/mL, or 0.5, 1, 2, 2.5, 3, 3.5, 4, 4.5, 4.6, 4.7, 4.8, 4.9, 5, 5.1, 5.2, 5.3, 5.4, 5.5, 6, 6.5, 7, 7.5, 8, 8.5, 9, 9.5, 10, 15, 20 or 25 mg/mL. In some embodiments, Compound 1 is present in the injectable composition in an amount of 1 to 10 mg/mL. In some embodiments, Compound 1 is present in the injectable composition in an amount of 4 to 6 mg/mL. In some embodiments, Compound 1 is present in the injectable composition in an amount of about 5 mg/mL.

The injectable composition also includes cyclodextrin in any suitable amount from 5% to 50% w/v. The cyclodextrin can be present in the injectable composition in an amount of from 5% to 50% w/v, or 5 to 25, or 10% to 20% w/v. The cyclodextrin can be present in the injectable composition in an amount of about 5% w/v, or 6, 7, 8, 9, 10, 11, 12, 13, 14, 15, 16, 17, 18, 19, 20, 25, 30, 35, 40, 45, or 50% w/v. In some embodiments, the cyclodextrin is present in the injectable composition in an amount of 5% to 50% w/v. In some embodiments, the cyclodextrin is present in the injectable composition in an amount of 10% to 20% w/v. In some embodiments, the cyclodextrin is present in the injectable composition in an amount of about 15% w/v. In some embodiments, the betadex-sulfobutylether sodium is present in the injectable composition in an amount of about 15% w/v.

The injectable composition can include Compound 1 and the cyclodextrin in any suitable combination of amounts as described above. For example, The injectable composition can include Compound 1 in an amount from 0.1% to 5% w/v, 0.1 to 4, 0.1 to 3, 0.1 to 2, 0.1 to 1, 0.2 to 0.8, 0.3 to 0.7, or 0.4% to 0.6% w/v, and cyclodextrin in an amount of from 5% to 50% w/v, or 5 to 25, or 10% to 20% w/v. The injectable composition can include Compound 1 in an amount of about 0.1% w/v, or 0.2, 0.3, 0.4, 0.5, 0.6, 0.7, 0.8, 0.9, or about 1% w/v, and cyclodextrin in an amount of about 6, 7, 8, 9, 10, 11, 12, 13, 14, 15, 16, 17, 18, 19, 20, 25, 30, 35, 40, 45, or 50% w/v. In some embodiments, the injectable composition includes Compound 1 in an amount of 0.1% to 10% w/v, and cyclodextrin in an amount of 5% to 50% w/v. In some embodiments, the injectable composition includes Compound 1 in an amount of 0.1% to 1% w/v, and cyclodextrin in an amount of 10% to 20% w/v. In some embodiments, the injectable composition includes Compound 1 in an amount of about 0.5% w/v, and cyclodextrin in an amount of about 15% w/v. In some embodiments, the injectable composition includes Compound 1 in an amount of about 0.5% w/v, and betadex-sulfobutylether sodium in an amount of about 15% w/v.

In some embodiments, the injectable composition includes Compound 1 in an amount of 0.1% to 10% w/v, cyclodextrin in an amount from 10% to 20% w/v, and water safe for injection. In some embodiments, the injectable composition includes Compound 1 in an amount of about 0.5% w/v, cyclodextrin in an amount of about 15% w/v, and water safe for injection. In some embodiments, the injectable composition consists essentially of Compound 1 in an amount of about 0.5% w/v, cyclodextrin in an amount of about 15% w/v, and water safe for injection.

In some embodiments, the injectable composition includes Compound 1 in an amount of 0.10% to 10% w/v, betadex-sulfobutylether sodium in an amount from 10% to 20% w/v, and water safe for injection. In some embodiments, the injectable composition includes Compound 1 in an amount of about 0.5% w/v, betadex-sulfobutylether sodium in an amount of about 15% w/v, and water safe for injection. In some embodiments, the injectable composition consists essentially of Compound 1 in an amount of about 0.5% w/v, betadex-sulfobutylether sodium in an amount of about 15% w/v, and water safe for injection.

The injectable composition can be contained in any suitable container, such as a sealed vial. In some embodiments, the present invention provides a sealed vial containing the injectable composition. In some embodiments, the present invention provides a sealed vial containing the injectable composition consisting essentially of Compound 1 in an amount of about 0.5% w/v, cyclodextrin in an amount of about 15% w/v, and water safe for injection.

### IV. Methods of Treating

In some embodiments, the present invention provides a method of treating a viral infection comprising administering a pharmaceutical composition comprising Compound 1, or a pharmaceutically acceptable salt thereof, cyclodextrin, and, optionally, pH adjusting agents. In some embodiments, the pharmaceutical composition is a composition of the present invention.

In some embodiments, the present invention provides a method of treating a viral infection comprising administering a pharmaceutical composition comprising Compound 1, or a pharmaceutically acceptable salt thereof, and beta-cyclodextrin, and, optionally, pH adjusting agents. In some embodiments, the pharmaceutical composition is a composition of the present invention.

In some embodiments, the present invention provides a method of treating a viral infection comprising parenterally administering a pharmaceutical composition comprising Compound 1, or a pharmaceutically acceptable salt thereof, and beta-cyclodextrin, and, optionally, pH adjusting agents. In some embodiments, the pharmaceutical composition is a composition of the present invention.

In some embodiments, the present invention provides a method of treating a viral infection comprising (a) reconstituting with safe water for injection a lyophilized pharmaceutical composition comprising Compound 1, or a pharmaceutically acceptable salt thereof, and beta-cyclodextrin, and, optionally, pH adjusting agents and (b) administering the reconstituted pharmaceutical composition. In some embodiments, the pharmaceutical composition is a composition of the present invention. In some embodiments, the reconstituted pharmaceutical composition is an injectable composition of the present invention. In some embodiments, the lyophilized pharmaceutical composition is a lyophilized composition of the present invention.

In some embodiments, the present invention provides a method of treating a viral infection comprising (a) reconstituting with safe water for injection a lyophilized pharmaceutical composition comprising Compound 1, or a pharmaceutically acceptable salt thereof, and beta-cyclodextrin, and, optionally, pH adjusting agents and (b) parenterally administering the reconstituted pharmaceutical composition. In some embodiments, the pharmaceutical composition is a composition of the present invention. In some embodiments, the reconstituted pharmaceutical composition is an injectable composition of the present invention. In some embodiments, the lyophilized composition is a lyophilized pharmaceutical composition of the present invention.

In some embodiments, the parenteral administration is selected from the group consisting of intramuscular (IM), subcutaneous (SC) and intravenous (IV) administrations.

In some embodiments, the present invention provides a method of treating a virus comprising parenterally administering a pharmaceutical composition comprising Compound 1, or a pharmaceutically acceptable salt thereof, and beta-cyclodextrin, and, optionally, pH adjusting agents. In some embodiments, the present invention provides a method of treating a virus comprising parenterally administering a pharmaceutical composition comprising Compound 1, or a pharmaceutically acceptable salt thereof, and beta-cyclodextrin, and, optionally, pH adjusting agents, wherein the virus is an Arenaviridae virus, a Coronaviridae virus, a Filoviridae virus, a Flaviviridae virus or a Paramyxoviridae virus. In some embodiments, the present invention provides a method of treating a virus comprising parenterally administering a pharmaceutical composition comprising Compound 1, or a pharmaceutically acceptable salt thereof, and beta-cyclodextrin, and, optionally, pH adjusting agents, wherein the virus is Lassa, Junin, Severe Acute Respiratory Syndrome (SARS), Middle Eastern Respiratory Syndrome (MERS), ebolavirus, Marburg virus, Zika, or Respiratory Syncytial virus. In some embodiments, the present invention provides a method of treating a virus comprising parenterally administering a pharmaceutical composition comprising Compound 1, or a pharmaceutically acceptable salt thereof, and beta-cyclodextrin, and, optionally, pH adjusting agents, wherein the virus is Lassa, Junin, Severe Acute Respiratory Syndrome (SARS), Middle Eastern Respiratory Syndrome (MERS), other human coronaviruses (229E, NL63, OC43, HKU1, or WIV1), zoonotic coronaviruses (PEDV or HKU CoV isolates such as HKU3, HKU5, or HKU9), ebolavirus (variants Zaire, Bundibugio, Sudan, Tai forest, or Reston), Marburg virus, Nipah, Hendra, Measles, Mumbs, Dengue, Yellow Fever, West Nile Virus, Zika, Parainfluenza, Metapneumovirus or Respiratory Syncytial virus.

In some embodiments, the present invention provides a method of treating a virus comprising parenterally administering a pharmaceutical composition comprising Compound 1, or a pharmaceutically acceptable salt thereof, and beta-cyclodextrin, and, optionally, pH adjusting agents, wherein the virus is ebolavirus.

### Combination Therapies

In some embodiments, the present invention provides a method for treating an Arenaviridae virus, a Coronaviridae virus, a Filoviridae virus, a Flaviviridae virus or a Paramyxoviridae virus infection in a human comprising administering to the human a therapeutically effective amount of a composition disclosed herein in combination with a therapeutically effective amount of one or more (e.g., one, two, three, one or two, or one to three) additional therapeutic agents. In some embodiments, the present invention provides a method for treating an Arenaviridae virus infection in a human comprising administering to the human a therapeutically effective amount of a composition disclosed herein in combination with a therapeutically effective amount of one or more (e.g., one, two, three, one or two, or one to three) additional therapeutic agents. In some embodiments, the present invention provides a method for treating a Coronaviridae virus infection in a human comprising administering to the human a therapeutically effective amount of a composition disclosed herein in combination with a therapeutically effective amount of one or more (e.g., one, two, three, one or two, or one to three) additional therapeutic agents. In some embodiments, the present invention provides a method for treating a Filoviridae virus infection in a human comprising administering to the human a therapeutically effective amount of a composition disclosed herein in combination with a therapeutically effective amount of one or more (e.g., one, two, three, one or two, or one to three) additional therapeutic agents. In some embodiments, a method for treating a Flaviviridae virus infection in a human comprising administering to the human a therapeutically effective amount of a composition disclosed herein in combination with a therapeutically effective amount of one or more (e.g., one, two, three, one or two, or one to three) additional therapeutic agents. In some embodiments, the present invention provides a method for treating a Paramyxoviridae virus infection in a human comprising administering to the human a therapeutically effective amount of a composition disclosed herein in combination with a therapeutically effective amount of one or more (e.g., one, two, three, one or two, or one to three) additional therapeutic agents.

In some embodiments, the additional therapeutic agent used in combination with a compound disclosed herein is active against an Arenaviridae virus, a Coronaviridae virus, a Filoviridae virus, a Flaviviridae virus or a Paramyxoviridae virus infections. Non-limiting examples of these other active therapeutic agents are ribavirin, palivizumab, motavizumab, RSV-IGIV (RespiGam®), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), favipiravir (also known as T-705 or Avigan), T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), JK-05, TKM-Ebola, ZMapp, rNAPc2, VRC-EBOADC076-00-VP, OS-2966, MVA-BN filo, brincidofovir, Vaxart adenovirus vector 5-based ebola vaccine, Ad26-ZEBOV, FiloVax vaccine, GOVX-E301, GOVX-E302, ebola virus entry inhibitors (NPC1 inhibitors), and rVSV-EBOV, and mixtures thereof. The compounds and compositions of the present invention may also be used in combination with phosphoramidate morpholino oligomers (PMOs), which are synthetic antisense oligonucleotide analogs designed to interfere with translational processes by forming base-pair duplexes with specific RNA sequences. Examples of PMOs include AVI-7287, AVI-7288, AVI-7537, AVI-7539, AVI-6002, and AVI-6003.

In some embodiments, the present invention provides a method of treating a Filoviridae virus infections comprising administering a composition comprising Compound 1, or a pharmaceutically acceptable salt thereof, cyclodextrin, and, optionally, pH adjusting agents, in combination with an additional therapeutic agent wherein the additional therapeutic agent is ZMapp.

Co-administration of a composition disclosed herein with a therapeutically effective amount of a one or more additional therapeutic agents generally refers to simultaneous or sequential administration of a compound disclosed herein and one or more additional therapeutic agents, such that the composition disclosed herein and the one or more additional therapeutic agents are both present in the body of the patient.

Co-administration includes administration of unit dosages comprising a therapeutically effective amount of one or more compositions disclosed herein before or after administration of unit dosages of a therapeutically effective amount of one or more additional therapeutic agents, for example, administration of the compositions disclosed herein within seconds, minutes, or hours of the administration of one or more additional therapeutic agents. For example, in some embodiments, a unit dose of a compound disclosed herein is administered first, followed within seconds or minutes by administration of a unit dose of one or more additional therapeutic agents. Alternatively, in other embodiments, a unit dose of one or more additional therapeutic agents is administered first, followed by administration of a unit dose of a compound disclosed herein within seconds or minutes. In some embodiments, a unit dose of a compound disclosed herein is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound disclosed herein.

### V. Methods of Preparing

The compositions of the present invention can be prepared by means generally known to one of skill in the art for removing water from a substance. For example, the compositions can be dehydrated by heating the sample to a suitable temperature for a suitable period of time. The compositions can also be dehydrated under a reduced pressure atmosphere at any suitable temperature. The reduced pressure atmosphere can be any pressure less than atmospheric pressure. The reduced pressure atmosphere can be heated to a temperature above room temperature, be maintained at about room temperature, or cooled to a temperature below room temperature. For example, the compositions can be cooled to a temperature of less than room temperature while under a reduced pressure atmosphere. Suitable temperatures include, but are not limited to, less than room temperature, or less than 20° C., 15, 10, 5, 0, −5, −10, −15, −20, −25, −30, −40, or less than −50° C. When a reduced pressure atmosphere is used, the reduced pressure atmosphere can be less than atmospheric pressure, or less than 100 torr (mm Hg), 50, 25, 10, 5, 4.58 (the triple point for water), 4, 3, 2, 1, 0.5, 0.1, 0.05, or less than 0.01 torr. The compositions can be cooled to a temperature of less than 0.01° C. while under a reduced pressure atmosphere of less than 4.58 torr (611 Pascal or 0.006 atmospheres).

The compositions of the present invention can be prepared according to the methods described in the examples below and variations thereof understood by one of skill in the art. For example, the lyophilized composition of the present invention can be prepared by dissolving cyclodextrin in water, acidifying the cyclodextrin mixture to a pH of less than 2, adding Compound 1 to the acidified mixture to form a suspension of the complex of Compound 1 and the cyclodextrin, adjusting the pH of the complexed mixture as needed to a pH of about 2 to form a solution of the complex of Compound 1 and the cyclodextrin, adjusting the pH of the solution to about 3.5 to form a supersaturated solution, adding water to the supersaturated solution as needed to form a mixture wherein the Compound 1 is at a concentration of about 6.7 mg/mL and the cyclodextrin is at a concentration of about 20% w/v, and lyophilizing the mixture. In some embodiments, the present invention provides a method of preparing a lyophilized composition including Compound 1:

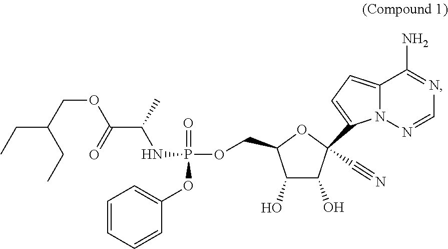 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

or a pharmaceutically acceptable salt thereof, and cyclodextrin, wherein the method includes forming a first mixture comprising cyclodextrin and water, such that the cyclodextrin dissolves; adding acid to the first mixture to form a second mixture having a pH of less than 2; adding Compound 1 to the second mixture, to form a third mixture; optionally adding acid or base to the third mixture to form a fourth mixture having a pH of from 3 to 4; and lyophilizing the third or fourth mixture to form the lyophilized composition.

### Compound 1

Compound 1 can be used in any suitable amount to achieve the desired concentration in mixture prior to lyophilization. For example, Compound 1 can be present in an amount of 1 mg to 1000 mg, or 5 to 500, or 50 to 250, or 60 to 240, or 70 to 230, or 80 to 220, or 90 to 210, or 100 to 200, or 90 to 110, or 145 to 165, or 90 mg to 175 mg. Compound 1 can also be present in an amount of about 10 mg, or about 20, 30, 40, 50, 60, 70, 80, 90, 100, 110, 120, 130, 140, 150, 160, 170, 180, 190, 200, 210, 220, 230, 240, or about 250 mg. In some embodiments, the mixture includes 90 mg to 175 mg of Compound 1. In some embodiments, the mixture includes 100 mg of Compound 1. In some embodiments, the mixture includes 150 mg of Compound 1.

In some embodiments, Compound 1 is present in the third or fourth mixture at about 1 to 10 mg/mL. In some embodiments, Compound 1 is present at about 4 to 8 mg/mL. In some embodiments, Compound 1 is present at about 5 to 7 mg/mL. In some embodiments, Compound 1 is present at about 5 mg/mL. In some embodiments, Compound 1 is present at about 6.7 mg/mL. In some embodiments, Compound 1 is present in the fourth mixture at about 6.7 mg/mL. In some embodiments, Compound 1 is present at 5.0 mg/mL. In some embodiments, Compound 1 is present at 6.7 mg/mL. In some embodiments, Compound 1 is present in the fourth mixture at 6.7 mg/mL.

Compound 1 can be of any suitable form. For example, Compound 1 can be amorphous or crystalline. In some embodiments, Compound 1 is amorphous Compound 1. Crystalline forms of Compound 1 useful in the methods and compositions of the present invention are described in U.S. application Ser. No. 15/964,597. For example, Compound 1 can be crystalline Form I, Form II, Form III or Form IV. In some embodiments, Compound 1 is crystalline Compound 1.

In some embodiments, Compound 1 is crystalline Compound 1 Form II. In some embodiments, crystalline Compound 1 is characterized by an X-ray powder diffraction (XRPD) pattern having at least three peaks selected from the group consisting of 22.3°, 16.2°, 22.5°, 13.8°, 12.7°, 16.9°, 10.6°, 14.5°, 24.3, 24.0°, 17.6°, 23.4°, 8.1°, 11.0°, 26.8°, 28.9°, 19.6°, 27.8°, 26.4°, 28.7°, 29.8°, 33.0°, 18.8°, 18.3°, 32.1°, 25.3°, 32.6°, 8.6°, 34.2°, 35.9°, 27.2°, 28.1°, 38.9°, 34.6°, 17.1°, 35.2°, 21.4°, 30.6°, 25.6°, 18.5°, 31.7°, 36.5°, and 37.1° 0.2° 2−θ.

In some embodiments, crystalline Compound 1 Form II has an XRPD pattern comprising degree 2θ-reflections (+/−0.2 degrees 2θ) at 22.3°, 16.9°, and 16.2°. In some embodiments, crystalline Compound 1 Form II has an XRPD pattern comprising degree 2θ-reflections (+/−0.2 degrees 2θ) at 22.3°, 16.9°, and 16.2° and one or more of the degree 2θ-reflections (+/−0.2 degrees 2θ) at 13.8° and 12.7°. In some embodiments, crystalline Compound 1 Form II has an XRPD pattern comprising degree 2θ-reflections (+/−0.2 degrees 2θ) at 22.3°, 16.9°, and 16.2° and one of the degree 2θ-reflections (+/−0.2 degrees 2θ) at 13.8° and 12.7°. In some embodiments, crystalline Compound 1 Form II has an XRPD pattern comprising degree 2θ-reflections (+/−0.2 degrees 2θ) at 22.3°, 16.9°, and 16.2° and two of the degree 2θ-reflections (+/−0.2 degrees 2θ) at 13.8° and 12.7°. In some embodiments, crystalline Compound 1 Form II has an XRPD pattern comprising degree 2θ-reflections (+/−0.2 degrees 2θ) at 22.3°, 16.9°, 16.2°, 13.8° and 12.7°. In some embodiments, crystalline Compound 1 Form II has an XRPD pattern comprising any three degree 2θ-reflections (+/−0.2 degrees 2θ) selected from the group consisting of 22.3°, 16.9°, 16.2°, 13.8°, and 12.7°.

In some embodiments, crystalline Compound 1 Form II has an XRPD pattern further comprising degree 2θ-reflections (+/−0.2 degrees 2θ) at 22.5°, 10.6° and 14.5°. In some embodiments, crystalline Compound 1 Form II has an XRPD pattern comprising degree 2θ-reflections (+/−0.2 degrees 2θ) at 22.3°, 16.9°, 16.2°, 13.8° and 12.7° and one or more of the degree 2θ-reflections (+/−0.2 degrees 2θ) at 22.5°, 10.6° and 14.5°. In some embodiments, crystalline Compound 1 Form II has an XRPD pattern comprising degree 2θ-reflections (+/−0.2 degrees 2θ) at 22.3°, 16.9°, 16.2°, 13.8° and 12.7° and one of the degree 2θ-reflections (+/−0.2 degrees 2θ) at 22.5°, 10.60 and 14.5°. In some embodiments, crystalline Compound 1 Form II has an XRPD pattern comprising degree 2θ-reflections (+/−0.2 degrees 2θ) at 22.3°, 16.9°, 16.2°, 13.8° and 12.7° and two of the degree 2θ-reflections (+/−0.2 degrees 2θ) at 22.5°, 10.6° and 14.5°. In some embodiments, crystalline Compound 1 Form II has an XRPD pattern comprising degree 2θ-reflections (+/−0.2 degrees 2θ) at 22.3°, 16.9°, 16.2°, 13.8°, 12.7°, 22.5°, 10.6° and 14.5°. In some embodiments, crystalline Compound 1 Form II has an XRPD pattern comprising any three degree 2θ-reflections (+/−0.2 degrees 2θ) selected from the group consisting of 22.3°, 16.9°, 16.2°, 13.8°, 12.7°, 22.5°, 10.6° and 14.5°.

Compound 1 can have any suitable purity. For example, the compound of Formula I can have a purity of at least 90%, or at least 91, 92, 93, 94, 95, 96, 97, 98, 99, or at least 99.1, 99.2, 99.3, 99.4, 99.5, 99.6, 99.7, 99.8 or at least 99.9%. In some embodiments, Compound 1 has a purity of at least 99.1%. In some embodiments, Compound 1 has a purity of at least 99.3%. In some embodiments, Compound 1 has a purity of at least 99.5%. In some embodiments, Compound 1 has a purity of at least 99.7%.

The impurities present in Compound 1 can include unreacted starting material, undesirable side-products, and other materials. Representative impurities include Impurity A:

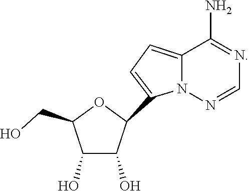 [SMILES: Nc1ncnn2c([C@@H]3O[C@H](CO)[C@@H](O)[C@H]3O)ccc12]

Impurity A can be present in an amount less than about 0.5%, or less than about 0.45%, 0.40, 0.35, 0.30, 0.25, 0.20, 0.15, 0.10, 0.09, 0.08, 0.07, 0.06, 0.05, 0.04, 0.03, 0.02, or less than about 0.01%. The amount of Impurity A can be measured in % AN as measured by HPLC, or can be based on weight (w/w). In some embodiments, Compound 1 includes less than 0.10% Impurity A. In some embodiments, Compound 1 includes less than 0.05% Impurity A.

In some embodiments, Compound 1 can have a purity of at least 99.1%, and include less than 0.10% Impurity A. In some embodiments, Compound 1 can have a purity of at least 99.1%, and include less than 0.05% Impurity A. In some embodiments, Compound 1 can have a purity of at least 99.1%, and include less than 0.04% Impurity A. In some embodiments, Compound 1 can have a purity of at least 99.5%, and include less than 0.04% Impurity A. In some embodiments, Compound 1 can have a purity of at least 99.5%, and include less than 0.04% Impurity A.

### Cyclodextrin

The cyclodextrin can include any suitable cyclodextrin as described above. For example, the cyclodextrin can be a beta-cyclodextrin, such as sulfobutylalkylether-beta-cyclodextrin, betadex-sulfobutylether sodium, or hydroxypropyl-beta-cyclodextrin. In some embodiments, the cyclodextrin includes a beta-cyclodextrin. In some embodiments, the cyclodextrin includes sulfobutylalkylether-beta-cyclodextrin, betadex-sulfobutylether sodium, or hydroxypropyl-beta-cyclodextrin. In some embodiments, the cyclodextrin includes betadex-sulfobutylether sodium.

The cyclodextrin can be present in any suitable amount. For example, the cyclodextrin can be present in the mixture at 1 to 50% w/v, or 5 to 30, or 10 to 25, or 14% to 21% w/v. The cyclodextrin can also be present in the mixture at about 1% w/v, or 5, 10, 11, 12, 13, 14, 15, 16, 17, 18, 19, 20, 21, 22, 23, 24, 25, 30, 35, 40, 45 or about 50% w/v. In some embodiments, the cyclodextrin is present at 5% to 30% w/v. In some embodiments, the cyclodextrin is present at 10% to 25% w/v. In some embodiments, the cyclodextrin is present at 14% to 21% w/v. In some embodiments, the cyclodextrin is present at about 15% w/v. In some embodiments, the cyclodextrin is present at about 20% w/v. In some embodiments, the cyclodextrin is present at 15% w/v. In some embodiments, the cyclodextrin is present at 20% w/v.

In some embodiments, the beta-cyclodextrin is present at about 5% to 30% w/v. In some embodiments, the beta-cyclodextrin is present at 10% to 25% w/v. In some embodiments, the beta-cyclodextrin is present at 14% to 21% w/v. In some embodiments, the beta-cyclodextrin is present at about 15% w/v. In some embodiments, the beta-cyclodextrin is present at about 20% w/v. In some embodiments, the beta-cyclodextrin is present at 15% w/v. In some embodiments, the beta-cyclodextrin is present at 20% w/v.

### pH Adjusting Agents

The pH adjusting agents useful in the methods of the present invention include any suitable acid and/or base. Representative acids include mineral acids such as hydrofluoric acid, hydrochloric acid, hydrobromice acid, etc. In some embodiments, the acid is hydrochloric acid. Other acids are useful in the methods of the present invention and are described above.

Representative bases include hydroxides such as lithium hydroxide, sodium hydroxide, potassium hydroxide, rubidium hydroxide, cesium hydroxide, magnesium hydroxide, strontium hydroxide, barium hydroxide, and others. In some embodiments, the base is sodium hydroxide. Other bases are useful in the methods of the present invention and are described above.

The pH adjusting agents can be used in any suitable amount to adjust the pH of the mixture to achieve the target pH.

In some embodiments, the mixture includes Compound 1, or a pharmaceutically acceptable salt thereof, and beta-cyclodextrin, and, optionally, pH adjusting agents, wherein Compound 1 is present at about 4.0 to 8.0 mg/mL and the beta-cyclodextrin is present at about 5% to 30% w/v. In some embodiments, the mixture includes Compound 1, or a pharmaceutically acceptable salt thereof, and beta-cyclodextrin, and, optionally, pH adjusting agents, wherein Compound 1 is present at about 4.0 to 8.0 mg/mL and the beta-cyclodextrin is present at about 10% to 25% w/v. In some embodiments, the mixture includes Compound 1, or a pharmaceutically acceptable salt thereof, and beta-cyclodextrin, and, optionally, pH adjusting agents, wherein Compound 1 is present at about 4.0 to 8.0 mg/mL and the beta-cyclodextrin is present at about 14% to 21% w/v.

In some embodiments, the mixture includes Compound 1, or a pharmaceutically acceptable salt thereof, and beta-cyclodextrin, and, optionally, pH adjusting agents, wherein Compound 1 is present at about 5.0 to 7.0 mg/mL and the beta-cyclodextrin is present at about 5% to 30% w/v. In some embodiments, the mixture includes Compound 1, or a pharmaceutically acceptable salt thereof, and beta-cyclodextrin, and, optionally, pH adjusting agents, wherein Compound 1 is present at about 5.0 to 7.0 mg/mL and the beta-cyclodextrin is present at about 10% to 25% w/v. In some embodiments, the mixture includes Compound 1, or a pharmaceutically acceptable salt thereof, and beta-cyclodextrin, and, optionally, pH adjusting agents, wherein Compound 1 is present at about 5.0 to 7.0 mg/mL and the beta-cyclodextrin is present at about 14% to 21% w/v.

In some embodiments, the mixture includes Compound 1, or a pharmaceutically acceptable salt thereof, and beta-cyclodextrin, and, optionally, pH adjusting agents, wherein Compound 1 is present at about 5.0 mg/mL and the beta-cyclodextrin is present at about 15% w/v. In some embodiments, the mixture includes Compound 1, or a pharmaceutically acceptable salt thereof, and beta-cyclodextrin, and, optionally, pH adjusting agents, wherein Compound 1 is present at about 6.7 mg/mL and the beta-cyclodextrin is present at about 20% w/v.

In some embodiments, a vial comprising Compound 1, or a pharmaceutically acceptable salt thereof, and beta-cyclodextrin, and, optionally, pH adjusting agents, wherein Compound 1 is present at about 5.0 mg/mL and the beta-cyclodextrin is present at about 15% w/v further comprising water for injection. In some embodiments, a vial comprising Compound 1, or a pharmaceutically acceptable salt thereof, and beta-cyclodextrin, and, optionally, pH adjusting agents, wherein Compound 1 is present at about 6.7 mg/mL and the beta-cyclodextrin is present at about 20% w/v further comprising water for injection.

In some embodiments, the mixture includes about 90 to 175 mg Compound 1, or a pharmaceutically acceptable salt thereof, and beta-cyclodextrin, and, optionally, pH adjusting agents, wherein the beta-cyclodextrin is present at about 15 to 20% w/v. In some embodiments, the mixture includes about 100 mg Compound 1, or a pharmaceutically acceptable salt thereof, and beta-cyclodextrin, and, optionally, pH adjusting agents, wherein the beta-cyclodextrin is present at about 15 to 20% w/v. In some embodiments, the mixture includes about 150 mg Compound 1, or a pharmaceutically acceptable salt thereof, and beta-cyclodextrin, and, optionally, pH adjusting agents, wherein the beta-cyclodextrin is present at about 15 to 20% w/v.

### Process Conditions

The pH of the second mixture can be any suitable pH less than 2. For example, the pH of the second mixture can be less than 2, or 1.95, 1.9, 1.85, 1.8, 1.75, 1.7, 1.65, 1.6, or less than 1.5. The pH of the second mixture can be about 1.95, 1.9, 1.85, 1.8, 1.75, 1.7, 1.65, 1.6, or about 1.5. In some embodiments, the second mixture has a pH of about 1.75.

The pH of the third or fourth mixture can be any suitable pH from 3 to 4. For example, the pH of the third or fourth mixture can be about 3.1, 3.2, 3.3, 3.4, 3.5, 3.6, 3.7, 3.8, or about 3.9. In some embodiments, the fourth mixture has a pH of 3 to 4. In some embodiments, the fourth mixture has a pH of about 3.5.

In some embodiments, the method of preparing a lyophilized composition includes forming the first mixture comprising cyclodextrin and water, such that the cyclodextrin dissolves; adding acid to the first mixture to form a second mixture having a pH of less than 2; adding Compound 1 to the second mixture such that Compound 1 dissolves, to form the third mixture; adding base to the third mixture to form the fourth mixture having a pH of from 3 to 4; and lyophilizing the fourth mixture to form the lyophilized composition.

In some embodiments, the method of preparing a lyophilized composition includes forming the first mixture comprising betadex-sulfobutylether sodium and water, such that the betadex-sulfobutylether sodium dissolves; adding hydrochloric acid to the first mixture to form a second mixture having a pH of less than 2; adding Compound 1 to the second mixture such that Compound 1 dissolves, to form the third mixture; adding sodium hydroxide to the third mixture to form the fourth mixture having a pH of about 3.5; and lyophilizing the fourth mixture to form the lyophilized composition.

In some embodiments, the method of preparing a lyophilized composition includes forming the first mixture comprising betadex-sulfobutylether sodium and water, such that the betadex-sulfobutylether sodium dissolves; adding hydrochloric acid to the first mixture to form a second mixture having a pH of less than 2; adding a crystalline form of Compound 1 to the second mixture such that Compound 1 dissolves, to form the third mixture; adding sodium hydroxide to the third mixture to form the fourth mixture having a pH of about 3.5, wherein the fourth mixture is a supersaturated solution of Compound 1; and lyophilizing the fourth mixture to form the lyophilized composition.

In some embodiments, the method includes forming the first mixture comprising betadex-sulfobutylether sodium and water; adding hydrochloric acid to the first mixture to form the second mixture having a pH of less than 2; adding the crystalline form of Compound 1 in the second mixture, to form the third mixture; adding sodium hydroxide to the third mixture to form the fourth mixture having a pH of about 3.5; adding water to the fourth mixture such that Compound 1 is present at about 6.7 mg/mL and betadex-sulfobutylether sodium is present at about 20% w/v; and lyophilizing the fourth mixture to form the composition.

Betadex sulfobutyl ether sodium was dissolved in a suitable amount of water for injection in a suitable mixing vessel. The bulk solution was acidified by addition of HCl. Compound 1 was then added and the pH reduced with additional HCl and/or NaOH to facilitate Compound 1 dissolution. The pH of the bulk solution was then adjusted to a range of 3 to 4 by addition of HCl and/or NaOH to obtain an aqueous solution containing a supersaturated solution of Compound 1. Water for injection was then added to the solution to obtain the desired concentration of Compound 1 for vial filling followed by additional pH adjustment with HCl and/or NaOH if necessary. The solution was then sterilized via filtration and aseptically filled into glass vials and lyophilized to obtain a lyophilized powder for reconstitution.

### VI. Methods of Use

The use of beta cyclodextrin derivatives in formulating certain medicinal agents to improve solubility, safety and other parameters has been disclosed in, for example, U.S. patents U.S. Pat. Nos. 5,134,127, 5,376,645, 7,635,773, 7,625,878, and 8,410,077 the entirety of each of which is incorporated herein by reference. Prior to applicants' disclosure, the difficulty of preparing an IV solution of Compound 1 was unknown. Prior to applicants' disclosure the type of solubilizing agent(s) useful to achieve an effective IV formulation comprising Compound 1 was unknown. Applicants have surprisingly discovered that the use of beta-cyclodextrin derivatives is useful to achieve an effective IV solution of Compound 1. An IV solution of Compound 1 comprising a beta cyclodextrin derivative provides advantages of (1) ability to deliver varying required doses of Compound 1; (2) ability to adjust infusion rate without precipitation of Compound 1; (3) ability to avoid the problems associated with the use of other IV formulations such as the use of TWEEN® 80 or organic co-solvents.

As a result of the discovery disclosed herein, one of ordinary skill in the art is able to (1) prepare an IV solution of Compound 1 as a bolus solution (i.e. for direct use; or as a pre-concentrate for dilution prior to use in a human patient in need thereof). Thus, the present disclosure also provides the use of a kit comprising Compound 1 and a beta cyclodextrin derivative in an IV formulation for use in a human patient in need thereof. The kit may comprise a premixed large volume parenteral (LVP) or small volume parenteral SVP bag comprising Compound 1 and a beta cyclodextrin derivative. Alternatively, the kit may comprise an aqueous solution of a beta cyclodextrin (e.g. Captisol® or Dexolve-7®) and a vial of Compound 1 to be mixed on site or prior to use by one of ordinary skill in the art. The solution comprising a saline solution of a beta cyclodextrin e.g. Captisol® may need to be further diluted (e.g. use of about a 20-50% concentrate) by one of ordinary skill in the art prior to mixing to form an intravenous formulation for administration to a patient in need thereof. The present disclosure is thus directed to the preparation, manufacture and/or use of an intravenous formulation comprising Compound 1 and a beta cyclodextrin derivative for use in IV treatment. A preferred beta cyclodextrin derivative is Captisol®. Also preferred is the beta cyclodextrin derivative Dexolve-7®. Also preferred is the beta cyclodextrin derivative betadex sulfobutyl ether sodium. Thus, in some embodiments, the present disclosure provides a kit comprising Compound 1 and a beta-cyclodextrin derivative for the treatment of a Filoviridae virus infection in a patient in need thereof. The ultimate decision on dosing rate, concentration of beta cyclodextrin derivative solution to be dosed and duration thereof are to be made by a qualified caregiver.

### VII. EXAMPLES

### Example 1. Preparation of Lyophilized Composition

### Aqueous

Having determined the water solubility of Compound 1 is low at about 0.03 mg/mL, exploration of dissolving 1 to 300 mg of Compound 1 in water or saline may be impractical for use in treating Arenaviridae, Coronaviridae, Filoviridae, Flaviviridae, or Paramyxoviridae viral infections.

### Co-Solvents

In a co-solvent system, drug solubility decreases logarithmically as the percent of the solvent decreases linearly. Thus, there is potential for precipitation upon dilution, which may make a co-solvent system impractical for use in treating Arenaviridae, Coronaviridae, Filoviridae, Flaviviridae, or Paramyxoviridae viral infections.

### Pre-Concentrate

Pre-concentrated formulations of a surfactant and an organic solvent (pre-concentrates) were explored. A solution formulation comprising Compound 1 present in about 30 mg/mL in 25% TWEEN® 80, 50% PEG 300, and 25% water was evaluated, but showed precipitation upon 10× and 100× dilution in normal saline. Other explored formulations included solution formulations comprising Compound 1 present in about 5 mg/mL in 25% Tween 80, 25% PEG 300, 50% water and another solution formulation comprising Compound 1 present at about 5 mg/mL in 25% TWEEN® 80, 75% water. These other formulations did not exhibit precipitation upon dilution in normal saline, but would require larger amounts of TWEEN® 80 which could be associated with toxicity.

### Complexation

Complexation with a beta-cyclodextrin derivative was explored successfully. In a solution that utilizes complexation with beta cyclodextrin derivative, the drug solubility decreases linearly (assuming a 1:1 complex) as the amount of cyclodextrin decreases linearly, limiting the potential for precipitation of Compound 1 upon dilution.

However, in another experiment, 4.5 g of betadex sulfobutyl ether sodium was dissolved in a suitable amount of water for injection in a suitable mixing vessel. The bulk solution was acidified to a pH less than 2 by the addition of HCl to facilitate dissolution of Compound 1 and complexation with betadex sulfobutyl ether sodium. A crystalline form of Compound 1 (150 mg) was added. To make the pH more neutral to between 3 and 4, NaOH was added. Surprisingly, upon addition of NaOH, Compound 1 and betadex sulfobutyl ether sodium remained complexed. When molecules are complexed under an acidic condition, increasing the pH can result in weakened or broken complexation. However, complexation between Compound 1 and betadex sulfobutyl ether sodium was maintained, forming a supersaturated solution. After complexation and pH adjusting, the solution was sterilized via filtration and aseptically filled into glass vials having a concentration for Compound 1 of 6.7 mg/mL and 20% w/v for betadex sulfobutyl ether sodium. The mixture was lyophilized to obtain a lyophilized powder for reconstitution, having 3.23% (w/w) Compound 1 and 96.77% (w/w) betadex-sulfobutylether sodium.

### Stability Testing

Lyophilized drug product vials were placed into temperature and humidity controlled chambers for predefined time intervals and removed for testing. The drug product vials were tested using analytical methods determined to be stability indicating such as liquid chromatography to monitor product purity.

TABLE 1
18-Month Stability Data for Lyophilized Composition

| | | 18 Months | | 6 Months |
|---|---|---|---|---|
| | | 25° C./ | 30° C./ | 40° C./ |
| TEST | T = 0 | 60% RH | 75% RH | 75% RH |
| pH | 3.7 | 3.6 | 3.6 | 3.6 |
| Strength (%) | 100.0 | 100.1 | 100.3 | 99.2 |
| Deg Product | 0.0 | 0.2 | 0.2 | 0.3 |
| Content (%) | | | | |
| Water Content | 1.9 | 1.5 | 1.6 | 1.7 |
| Impurity A | 0.10 (Trace) | 0.10 | 0.10 | 0.10 |
| Impurity B | 0.08 (Trace) | 0.11 | 0.14 | 0.15 |

* ND < 0.05%.

### Example 2. Preparation of Injectable Composition

The lyophilized composition of Example 1, was reconstituted with 29 mL of sterile water for injection, to prepare a 5 mg/mL composition having 0.5% (w/v) of Compound 1 and 15% (w/v) of betadex-sulfobutylether sodium. The reconstituted solution was diluted into intravenous infusion fluids prior to intravenous infusion.

TABLE 2
24-Month Stability Data for Injectable Composition

| | | | | 6 M |
|---|---|---|---|---|
| | | 24 M | 12 M | 25° C./ |
| TEST | T = 0 | −20° C. | 5° C. | 60% RH |
| pH | 3.5 | 3.5 | 3.5 | 3.5 |
| Strength (%) | 98.8 | 97.4 | 96.5 | 88.3 |
| Impurity | 0.5 | 0.5 | 1.6 | 8.6 |
| Content (%) | | | | |
| Particulate | <657 | <657 | <657 | <657 |
| Matter | <171 | <171 | <171 | <171 |
| Impurity A | ND | ND | 0.07 (Trace) | 0.52 |
| Impurity B | ND | 0.06 (Trace) | 1.11 | 6.88 |

* ND < 0.05%.

Each of the references including all patents, patent applications and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application. Each of the references including all patents, patent applications and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A method of treating a viral infection in a human in need thereof, the method comprising
(a) reconstituting with safe water for injection a lyophilized pharmaceutical composition comprising: 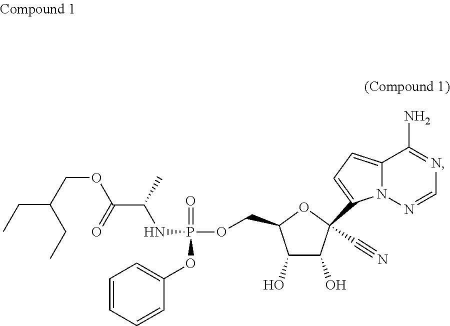 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]
or a pharmaceutically acceptable salt thereof, and
cyclodextrin;
wherein the lyophilized pharmaceutical composition comprises less than about 1% of impurity via HPLC following storage for 18 months at a temperature of 25° C. (±1° C.) and a relative humidity of 60% (±1%);
(b) administering to the human a therapeutically effective amount of the reconstituted pharmaceutical composition:
wherein the viral infection is caused by an Arenaviridae virus, a Coronaviridae virus, a Filoviridae virus, or a Paramxoviridae virus.

2. The method of claim 1, wherein the cyclodextrin is a beta-cyclodextrin.

3. The method of claim 2, wherein the beta-cyclodextrin is sulfobutylalkylether-beta-cyclodextrin, betadex-sulfobutylether sodium, or hydroxypropyl-beta-cyclodextrin.

4. The method of claim 2, wherein the beta-cyclodextrin is betadex-sulfobutylether sodium.

5.-12. (canceled)

13. The method of claim 1, wherein the lyophilized pharmaceutical composition comprises less than about 0.5% of impurity via HPLC.

14.-39. (canceled)

40. The method of claim 1, wherein the lyophilized pharmaceutical composition further comprises a pH adjusting agent.

41. The method of claim 40, wherein the pH adjusting agent comprises HCl, NaOH, or a combination thereof.

42. The method of claim 40, wherein the pH adjusting agent is HCl.

43. The method of claim 40, wherein the pH adjusting agent is NaOH.

44. The method of claim 1, wherein the lyophilized pharmaceutical composition has a pH of about 3 to about 4.

45. The method of claim 40, wherein the lyophilized pharmaceutical composition comprises
Compound 1 in an amount from 1% to 10% w/w; and
cyclodextrin in an amount from 90% to 99% w/w.

46. The method of claim 40, wherein the lyophilized pharmaceutical composition comprises
Compound 1 in an amount from 1% to 5% w/w; and
cyclodextrin in an amount from 95% to 99% w/w.

47. The method of claim 40, wherein the lyophilized pharmaceutical composition comprises
Compound 1 in an amount from 2% to 4% w/w; and
cyclodextrin in an amount from 96% to 98% w/w.

48. The method of claim 40, wherein the lyophilized pharmaceutical composition comprises
Compound 1 in an amount from 3% to 3.5% w/w; and
cyclodextrin in an amount from 96.5% to 97% w/w.

49. The method of claim 40, wherein the lyophilized pharmaceutical composition comprises
Compound 1 in an amount of about 3.2% w/w; and
betadex-sulfobutylether sodium in an amount of about 96.8% w/w.

50. The method of claim 40, wherein the lyophilized pharmaceutical composition consists essentially of
Compound 1 in an amount of about 3.2% w/w; and
betadex-sulfobutylether sodium in an amount of about 96.8% w/w.

51. The method of claim 1, wherein the viral infection is caused by a Coronaviridae virus.

52. The method of claim 1, wherein the administering further comprises one or more additional therapeutic agents.

53. A method of treating a viral infection in a human in need thereof, the method comprising
(a) reconstituting with safe water for injection a lyophilized pharmaceutical composition comprising: 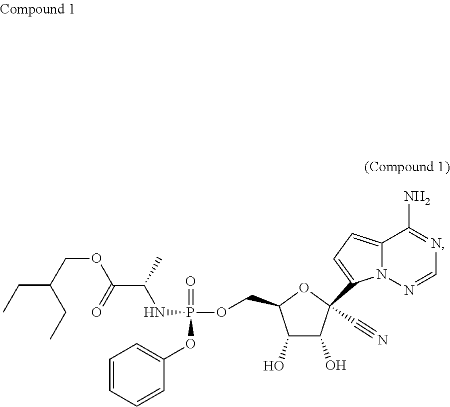 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]
or a pharmaceutically acceptable salt thereof, and
cyclodextrin;
wherein the lyophilized pharmaceutical composition comprises less than about 1% of impurity via HPLC following storage for 18 months at a temperature of 30° C. (±1° C.) and a relative humidity of 75% (±1%)
(b) administering to the human a therapeutically effective amount of the reconstituted pharmaceutical composition;
wherein the viral infection is caused by an Arenaviridae virus, a Coronaviridae virus, a Filoviridae virus, or a Paramyxoviridae virus.

54. The method of claim 53, wherein the cyclodextrin is a beta-cyclodextrin.

55. The method of claim 54, wherein the beta-cyclodextrin is sulfobutylalkylether-beta-cyclodextrin, betadex-sulfobutylether sodium, or hydroxypropyl-beta-cyclodextrin.

56. The method of claim 54, wherein the beta-cyclodextrin is betadex-sulfobutylether sodium.

57. The method of claim 53, wherein the lyophilized pharmaceutical composition comprises less than about 0.5% of impurity via HPLC.

58. The method of claim 53, wherein the lyophilized pharmaceutical composition further comprises a pH adjusting agent.

59. The method of claim 58, wherein the pH adjusting agent comprises HCl, NaOH, or a combination thereof.

60. The method of claim 58, wherein the pH adjusting agent is HCl.

61. The method of claim 58, wherein the pH adjusting agent is NaOH.

62. The method of claim 53, wherein the lyophilized pharmaceutical composition has a pH of about 3 to about 4.

63. The method of claim 58, wherein the lyophilized pharmaceutical composition comprises
Compound 1 in an amount from 1% to 10% w/w; and
cyclodextrin in an amount from 90% to 99% w/w.

64. The method of claim 58, wherein the lyophilized pharmaceutical composition comprises
Compound 1 in an amount from 1% to 5% w/w; and
cyclodextrin in an amount from 95% to 99% w/w.

65. The method of claim 58, wherein the lyophilized pharmaceutical composition comprises
Compound 1 in an amount from 2% to 4% w/w; and
cyclodextrin in an amount from 96% to 98% w/w.

66. The method of claim 58, wherein the lyophilized pharmaceutical composition comprises
Compound 1 in an amount from 3% to 3.5% w/w; and
cyclodextrin in an amount from 96.5% to 97% w/w.

67. The method of claim 58, wherein the lyophilized pharmaceutical composition comprises
Compound 1 in an amount of about 3.2% w/w; and
betadex-sulfobutylether sodium in an amount of about 96.8% w/w.

68. The method of claim 58, wherein the lyophilized pharmaceutical composition consists essentially of
Compound 1 in an amount of about 3.2% w/w; and
betadex-sulfobutylether sodium in an amount of about 96.8% w/w.

69. The method of claim 53, wherein the viral infection is caused by a Coronaviridae virus.

70. The method of claim 53, wherein the administering further comprises one or more additional therapeutic agents.

71. A method of treating a viral infection in a human in need thereof, the method comprising
(a) reconstituting with safe water for injection a lyophilized pharmaceutical composition comprising: 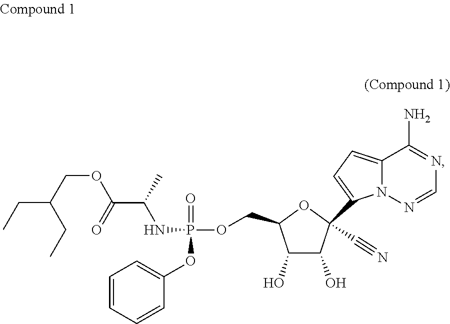 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]
or a pharmaceutically acceptable salt thereof, and
cyclodextrin;
wherein the reconstituted pharmaceutical composition comprises less than about 1% of impurity via HPLC following storage for 6 months at a temperature of 40° C. (±1° C.) and a relative humidity of 75% (±1%);
(b) administering to the human a therapeutically effective amount of the reconstituted pharmaceutical composition;
wherein the viral infection is caused by an Arenaviridae virus, a Coronaviridae virus, a Filoviridae virus, or a Paramyxoviridae virus.

72. The method of claim 71, wherein the cyclodextrin is a beta-cyclodextrin.

73. The method of claim 72, wherein the beta-cyclodextrin is sulfobutylalkylether-beta-cyclodextrin, betadex-sulfobutylether sodium, or hydroxypropyl-beta-cyclodextrin.

74. The method of claim 72, wherein the beta-cyclodextrin is betadex-sulfobutylether sodium.

75. The method of claim 71, wherein the lyophilized pharmaceutical composition comprises less than about 0.5% of impurity via HPLC.

76. The method of claim 71, wherein the lyophilized pharmaceutical composition further comprises a pH adjusting agent.

77. The method of claim 76, wherein the pH adjusting agent comprises HCl, NaOH, or a combination thereof.

78. The method of claim 76, wherein the pH adjusting agent is HCl.

79. The method of claim 76, wherein the pH adjusting agent is NaOH.

80. The method of claim 71, wherein the lyophilized pharmaceutical composition has a pH of about 3 to about 4.

81. The method of claim 76, wherein the lyophilized pharmaceutical composition comprises
Compound 1 in an amount from 1% to 10% w/w; and
cyclodextrin in an amount from 90% to 99% w/w.

82. The method of claim 76, wherein the lyophilized pharmaceutical composition comprises
Compound 1 in an amount from 1% to 5% w/w; and
cyclodextrin in an amount from 95% to 99% w/w.

83. The method of claim 76, wherein the lyophilized pharmaceutical composition comprises
Compound 1 in an amount from 2% to 4% w/w; and
cyclodextrin in an amount from 96% to 98% w/w.

84. The method of claim 76, wherein the lyophilized pharmaceutical composition comprises
Compound 1 in an amount from 3% to 3.5% w/w; and
cyclodextrin in an amount from 96.5% to 97% w/w.

85. The method of claim 76, wherein the lyophilized pharmaceutical composition comprises
Compound 1 in an amount of about 3.2% w/w; and
betadex-sulfobutylether sodium in an amount of about 96.8% w/w.

86. The method of claim 76, wherein the lyophilized pharmaceutical composition consists essentially of
Compound 1 in an amount of about 3.2% w/w; and
betadex-sulfobutylether sodium in an amount of about 96.8% w/w.

87. The method of claim 71, wherein the viral infection is caused by a Coronaviridae virus.

88. The method of claim 71, wherein the administering further comprises one or more additional therapeutic agents.
